(19) United States
(12) Patent Application Publication
(10) Pub. No.: US 2002/0048814 A1
(43) Pub. Date: Apr. 25, 2002

(54) Methods of gene silencing using poly-dT sequences

(75) Inventor: Paul Oeller, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: DNA Plant Technology Corporation, Oakland, CA (US)

(21) Appl. No.: 09/929,745

(22) Filed: Aug. 13, 2001

Related U.S. Application Data
(60) Provisional application No. 60/225,504, filed on Aug. 15, 2000.

(51) Int. Cl. A01H 5/00; C12N 15/82; C12N 15/86; C12N 15/87

(52) U.S. Cl. 435/455; 435/456; 435/468; 800/279

(57) ABSTRACT

The present invention provides methods for inhibiting target gene expression, by expressing in a cell a nucleic acid construct comprising a sense or antisense region having substantial sequence identity to a target gene, and a poly-dT sequence in a position 3′ to the sense or antisense region and 5′ of a polyadenylation signal.

### CROSS-REFERENCES TO RELATED APPLICATIONS

The present application claims the benefit of U.S. Ser. No. 60/225,504, filed Aug. 15, 2000, herein incorporated by reference in its entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Suppression of the expression of particular genes is an important tool both for research and for the development of genetically engineered organisms more fitted for a particular purpose. Gene silencing can be accomplished by the introduction of a transgene corresponding to the gene of interest in the antisense orientation relative to its promoter (see, e.g., Sheehy et al., Proc. Nat'l Acad. Sci. USA 85:8805-8808 (1988); Smith et al., Nature 334:724-726 (1988)), or in the sense orientation relative to its promoter (Napoli et al., Plant Cell 2:279-289 (1990); van der Krol et al., Plant Cell 2:291-299 (1990); U.S. Pat. No. 5,034,323; U.S. Pat. No. 5,231,020; and U.S. Pat. No. 5,283,184), both of which lead to reduced expression of the transgene as well as the endogenous gene.

Posttranscriptional gene silencing has been reported to be accompanied by the accumulation of small (20-25 nucleotide) fragments of antisense RNA, which have been reported to be synthesized from an RNA template and represent the specificity and mobility determinants of the process (Hamilton & Baulcombe, Science 286:950-952 (1999)). It has become clear that in a range of organisms the introduction of dsRNA (double-stranded RNA) is an important component leading to gene silencing (Fire et al., Nature 391:806-811 (1998); Timmons & Fire, Nature 395:854 (1998); WO99/32619; Kennerdell & Carthew, Cell 95:1017-1026 (1998); Ngo et al., Proc. Nat'l Acad. Sci. USA 95:14687-14692 (1998); Waterhouse et al., Proc. Nat'l Acad. Sci. USA 95:13959-13964 (1998); WO99/53050; Cogoni & Macino, Nature 399:166-169 (1999); Lohmann et al., Dev. Biol. 214:211-214 (1999); Sanchez-Alvarado & Newmark, Proc. Nat'l Acad. Sci. USA 96:5049-5054 (1999)). In plants the suppressed gene does not need to be an endogenous plant gene, since both reporter transgenes and virus genes are subject to posttranscriptional gene silencing by introduced transgenes (English et al., Plant Cell 8:179-188 (1996); Waterhouse et al, supra). However, in all of the above cases, some sequence similarity is required between the introduced transgene and the gene that is suppressed.

Previous reports of posttranscriptional gene silencing have used inverted repeat structures within the transgene expression construct (Hamilton et al., Plant J. 15:737-746 (1998); WO98/53083; Waterhouse et al., Proc. Nat'l Acad. Sci. USA 95:13959-13964 (1998)). Similar results were obtained by expression of sense and antisense transgenes under the control of different promoters in the same plant (Chuang & Meyerowitz, Proc. Nat'l Acad. Sci USA 97:4985-4990 (2000)).

As gene silencing is a powerful tool for regulation of gene expression, both of endogenous genes and of transgenes, improved methods of gene silencing are desired.

### SUMMARY OF THE INVENTION

The present invention provides an improved method for gene silencing that is specific for a target gene but does not require an inverted repeat nucleic acid anywhere in the transgene expression cassette. The method employs the incorporation of a poly-dT sequence positioned 5′ to the polyadenylation site of the transgene. Preferably, the poly-dT sequence is 3′ to the targeting sequence, although in some embodiments the poly-dT sequence is positioned 5′ to the targeting sequence. A silencing transgene expression cassette of the invention therefore comprises a promoter, a targeting sequence, and a poly-dT sequence, followed by a sequence which contains a recognition site for processing and polyadenylation of the 3′ end of the RNA. Messenger RNA produced from such a transgene can form a double-stranded region between the poly-U sequence, specified by the poly-dT sequence in the cassette, and the poly-A tail that is added by the normal polyadenylation process. Once the posttranscriptional gene silencing mechanism is triggered, sequences in cis to this region become targets of gene silencing. The poly-dT sequence is about 15 to about 2000 of base pairs in length, typically from about 50 to about 100-500 base pairs in length. This method has the advantage of ease and rapidity in preparation of the constructs, since a base vector can be designed to allow the insertion of any targeting sequence. The expression cassettes of the invention are also suitable for high throughput studies.

In one aspect, the present invention provides a method of reducing expression of a target gene in a eukaryotic cell, the method comprising the step of expressing in the cell an expression cassette comprising a promoter operably linked to a sense or antisense targeting sequence having substantial identity to at least a subsequence of the target gene, a poly-dT sequence, and a polyadenylation signal, wherein the poly-dT sequence is positioned 5′ to the polyadenylation signal, thereby reducing expression of the target gene.

In another aspect, the present invention provides an expression cassette comprising a promoter operably linked to a sense or antisense targeting sequence having substantial identity to at least a subsequence of the target gene, a poly-dT sequence, and a polyadenylation signal, wherein the poly-dT sequence is positioned 5′ to the polyadenylation signal.

In another aspect, the present invention provides a transgenic plant comprising an expression cassette comprising a promoter operably linked to a sense or antisense targeting sequence having substantial identity to at least a subsequence of the target gene, a poly-dT sequence, and a polyadenylation signal, wherein the poly-dT sequence is positioned 5′ to the polyadenylation signal.

In one embodiment, the poly-dT sequence is positioned 3′ or 5′ to the targeting sequence. In another embodiment, a linker sequence is present between the poly-dT sequence and the polyadenylation signal. In another embodiment, the poly-dT sequence is from about 50 to about 1000 nucleotides in length.

In one embodiment, the targeting sequence is a sense or an antisense sequence. In another embodiment, the targeting sequence has substantial identity to a plant pathogen target gene, e.g., a viral sequence, a bacterial sequence, an insect sequence, a fungal sequence, a parasitic sequence, or a nematode sequence. In another embodiment, the targeting sequence has substantial identity to a plant target gene. In another embodiment, the targeting sequence is from about 100 to about 1000 nucleotides in length. In another embodiment, the targeting sequence is from a coding region, a 5′ untranslated region, or a 3′ untranslated region of the target gene. In another embodiment, the targeting sequence comprises a premature stop codon that inhibits translation of the targeting sequence.

In one embodiment, the target gene is CONSTANS.

In one embodiment, the promoter is a tissue specific promoter. In another embodiment, the promoter is a plant promoter, e.g., a cauliflower mosaic virus 35S promoter or a figwort mosaic virus 34S promoter.

In one embodiment, the cell is a plant cell.

In another embodiment, the plant is selected from the group consisting of wheat, corn, rice, sorghum, pepper, tomato, squash, banana, strawberry, carrot, bean, cabbage, beet, cotton, grape, pea, pineapple, potato, soybean, yam, and alfalfa.

### BRIEF DESCRIPTION OF THE DRAWINGS

FIG. 1 provides a schematic representation of a poly-dT silencing construct. The poly-dT sequence is oriented such that the RNA transcript derived from the transgene contains a poly-U sequence 5′ to the poly-A tail.

### DETAILED DESCRIPTION OF THE INVENTION

Introduction

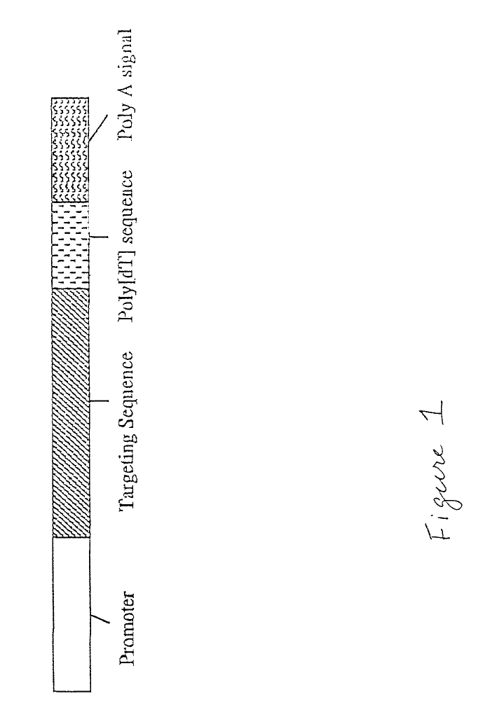

The present invention therefore provides improved methods of gene silencing, by expressing in an organism a nucleic acid having a poly-dT sequence 5′ to the polyadenylation signal of the transgene. Preferably, the poly-dT sequence is 3′ to the targeting sequence (see, e.g., FIG. 1). In some embodiment, the poly-dT sequence is 5′ to the targeting sequence. The poly-dT sequence is typically about 50 to about 100, 200, 300, 400, 500 or more base pairs in length and is oriented such that it specifies a poly-U sequence in the corresponding RNA transcript. The poly-U sequence of the mRNA can form a region of double-stranded RNA by pairing with the poly-A sequence added during the normal cellular process of mRNA 3′ end processing and maturation. Without being tied to theory, the dsRNA may form a hairpin or stem-loop structure. Optionally, a linker may be present between the poly-dT sequence and the 3′ polyadenylation signal, the linker typically being from about 15 to about 200-1000 base pairs in length, or longer.

The improved gene silencing construct is expressed in the organism of choice, e.g., a fungal cell, a eukaryotic cell, e.g., a plant cell or a mammalian cell. In one embodiment, the improved gene silencing construct is expressed in a plant cell, where the transcript, or fragments thereof, is taken up by plant pathogens such as fungi, bacteria, nematodes, e.g., cyst and root knot nematodes, and insects, e.g., sucking insects, leading to gene silencing in the pathogen. In another embodiment, the improved gene silencing construct is expressed in a transgenic plant, and is used to regulate expression of the transgene, e.g., in a hybrid plant vs. the parent plant, producing, e.g., male sterility. In another embodiment, the improved gene silencing construct is used in functional genomics to determine the effect of regulating gene expression of a selected endogenous gene or transgene. In another embodiment, the gene silencing vector is used to regulate expression of an endogenous plant gene, e.g., to regulate plant phenotypes such as disease resistance; modification of structural and storage polysaccharides; flavor; protein, nutritional characteristics; sugar, oil, and fatty acid composition; acidity; fruit ripening; fruit softening; yield; color/pigment; flowering; male sterility, etc. In another embodiment, the improved gene silencing construct is used to regulate multiple transgenes.

The target gene is any gene suitable for regulation in an organism. The gene may be an endogenous chromosomal or genomic gene, a transgene, either episomal or integrated, an episomal gene, a mitochondrial gene, a chloroplastic gene, a viral gene, either episomal or integrated, a bacterial gene, etc. For example, suitable targeting genes in plants include polygalacturonase, delta-12 desaturase, delta-9 desaturase, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, EPSP synthase. For example, in targeting a plant pathogen, genes involved in development, reproduction, motility, nervous system, sex determination, normal metabolic function and homeostasis, and the like, are suitable for targeting.

The construct is expressed by expression vectors comprising promoters active in the cells of choice, e.g., optionally constitutive or tissue specific promoters. For example, constitutive plant promoters include the cauliflower mosaic virus (CaMV) 35S promoter, the figwort mosaic virus (FMG) 34S promoter, and the 1′- or 2′-promoter derived from T-DNA of Agrobacterium tumefaciens. Examples of inducible plant promoters include promoters under developmental control that initiate transcription only in certain tissues, such as fruit, seeds, or flowers, or promoters that regulate transcription in response to environmental stimuli such as light or chemicals or pest infection, or promoters that are temporally regulated. For example, the use of a polygalacturonase promoter can direct expression in the fruit, a CHS-A (chalcone synthase A from petunia) promoter can direct expression in flower of a plant.

Other suitable promoters include, e.g., tapetal-specific promoters such as TA29 from tobacco (Mariani et al., Nature 347:737-41 (1990)), 127a, 108, and 92b from tomato (Chen & Smith, Plant Physiol. 101:1413-19 (1993); Aguirre & Smith, Plant Mol. Biol. 23:477-87 (1993)), and A6 and A9 from Brassica (Wyatt et al., Plant Mol. Biol. 19:611-22 (1992)). Anther-specific promoters could also be used such as ones isolated by Twell et al., Mol. Gen. Genet. 217:240-45 (1991) or Scott et al., Plant Mol. Biol. 17:195-207 (1991). Seed coat specific promoters, such as the pT218 promoter (Fobert et al., The Plant Journal 6:567-77 (1994)) or the pWM403 promoter could also be used in the present invention. Tissue-specific promoters for a range of different tissues have been identified, including roots, sepals, petals, and vascular elements. In addition, promoters induced upon pathogen infection have been identified, such as the prp-1 promoter (Strittmatter et al., Bio/Technology 13:1085-90 (1995)). Promoters induced in specialized nematode feeding structures have been identified (disclosed in patent applications WO 92/21757, WO 93/10251, WO 93/18170, WO 94/10320, WO 94/17194). Another useful promoter is the tet artificial promoter comprising at least one tet operators and a TATA-box (Weinman et al., 1994). This promoter is transcriptionally activated by an activator made by fusing the tet repressor, which recognizes the tet operator, to a eukaryotic activation domain.

Suitable expression vectors for use in the present invention include prokaryotic and eukaryotic vectors, include mammalian vectors and plant vectors. Plant vectors can include DNA or RNA expression vectors. For example, plant RNA expression vectors include derivatives of plant RNA viruses in the Bromovirus, Furovirus, Hordeivirus, Potexvirus, Tobamovirus, Tobravirus, Tombusvirus, and Potyvirus groups, in particular tobacco mosaic virus, cucumber mosaic virus, tobacco etch virus, tobacco rattle virus, tomato bushy stunt virus, brome mosaic virus, potato virus X, and potato virus Y. Suitable DNA expression vectors of the invention also include, e.g., viral-based vectors derived from plant DNA viruses, e.g., from Caulimovirus or Geminivirus, in particular, from cauliflower mosaic virus, African cassava mosaic virus, and tomato golden mosaic virus.

Suitable plants for use in the methods of the invention include a broad range of plants, including, e.g., species from the genera Allium, Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Musa, Nicotiana, Olea, Oryza, Panieum, Pannesetum, Persea, Phaseolus, Pistachia, Pisum, Pyrus, Prunus, Raphanus, Ricinus, Rosa, Secale, Senecio, Sinapis, Solanum, Sorghum, Theobromus, Trigonella, Triticum, Vicia, Vitis, Vigna, and Zea.

Definitions

The phrase “inhibiting expression of a target gene” refers to the ability of a nucleic acid construct of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, preferably 50%, more preferably 25-0%. Suitable assays include those described below in the Example section, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

A “target gene” refers to any gene suitable for regulation of expression, including both endogenous chromosomal genes and transgenes, as well as episomal or extrachromosomal genes, mitochondrial genes, chloroplastic genes, viral genes, bacterial genes, animal genes, plant genes, protozoal genes and fungal genes.

A “targeting sequence” refers to a nucleic acid that has substantial identity to the target gene and is part of the gene silencing vector. The targeting sequence can correspond to the full length target gene, or a subsequence thereof. Typically, the targeting sequence is at least about 25-50 nucleotides in length.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes (i.e., genes that do not have substantial identity to one another) arranged to make a transcribed nucleic acid, e.g., a coding region from another source and a poly-dT tract.

“Poly-dT sequence” refers to a nucleic acid sequence oriented in an expression cassette so that it produces a poly-U sequence in the corresponding RNA transcript. The poly-dT sequence is positioned 5′ to the polyadenylation signal in the expression cassette. The poly-dT sequence may optionally include a linker sequence between the sequence and the 3′ mRNA processing signal that leads to polyadenylation of the transcript. The poly-dT sequence has sufficient length to for a double stranded region with a poly-A tail; e.g., the poly-dT tract is typically 15-2000 nucleotides in length, preferably 50 to 500 nucleotides in length, or 100-300 nucleotides in length, or 150-200 nucleotides in length. The poly-dT sequence also has sufficient identity to a poly-A tract to form a double stranded region with a poly-A tail of a transcript, preferably 90% identity or higher.

A “polyadenylation signal or site” or a “3′ processing or maturation signal or site” refers to conserved cis acting sequences (e.g., a polyadenylation site such as AAUAAA or a variant thereof) that direct cellular proteins to process the 3′ end of an RNA and to polyadenylate the 3′ end of the RNA (see, e.g., Alberts et al., Molecular Biology of the Cell (3^{rd} ed. 1994); Twyman, Advanced Molecular Biology (1998)).

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions and in most plant tissues. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

A “plant promoter” is a promoter capable of initiating transcription in plant cells.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. The expression vector can be an RNA or a DNA vector. Typically, the expression vector includes a nucleic acid to be transcribed operably linked to a promoter (an expression cassette). An “expression cassette” is a subsequence of an expression vector.

The terms “substantially identical” or “substantial identity,” in the context of two or more nucleic acids or polypeptide sequences, refer to two or more sequences or subsequences that are the same or have a specified percentage of amino acid residues or nucleotides that are the same (i.e., at least about 60%, preferably 65%, 70%, 75%, preferably 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the identity exists over a region that is at least about 6-7 amino acids or at least about 25 nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Improved Gene Silencing Vectors

The improved gene silencing vectors disclosed herein can be used to inhibit target gene expression in an organism of choice, e.g., eukaryotes such as a plant or a plant pathogen, e.g., a nematode, a virus, an insect, etc., or a mammal. To accomplish this, a targeting nucleic acid sequence from the desired target gene is cloned and operably linked to a promoter or promoters such that either a sense and an antisense strand of RNA will be transcribed. A poly-dT sequence is positioned 5′ of the polyadenylation signal of the transgene, such that a poly-U sequence will be incorporated into the RNA transcript. Preferably, the poly-dT sequence is 3′ to the targeting region, although in some embodiments it is 5′ to the targeting region. The construct is then transformed into the organism of choice, and RNA is produced. The targeting nucleic acid sequence to be introduced generally will be substantially identical (i.e., have at least about a minimum percent identity) to at least a portion of the target gene or genes to be inhibited. This minimal identity will typically be at least about 60%, but a higher identity might exert a more effective repression of expression of the endogenous sequences. For maximum suppression, substantially greater identity of more than about 80% may preferred, and about 95% to absolute identity may be most preferred. The sequence, however, need not be perfectly identical to inhibit expression. The vectors of the present invention can be designed such that the inhibitory effect applies to other genes within a family of genes exhibiting identity or substantial identity to the target gene.

The introduced targeting sequence also need not be full length relative to either the primary transcription product or fully processed mRNA. Generally, higher identity can be used to compensate for the use of a shorter sequence. Furthermore, the introduced sequence need not have the same intron or exon pattern, and identity of non-coding segments may be equally effective. Normally, the targeting sequence has a length of at least about 25 nucleotides, optionally a sequence of about 25 to about 50 nucleotides, optionally a sequence of about 50 to about 100 nucleotides, optionally a sequence of about 100 to about 200 nucleotides, optionally a sequence of about 200 to about 500, and optionally a sequence of about 500 to about 2000 or more nucleotides, up to molecule that corresponds in size to the full length target gene.

Cloning of Target Nucleic Acids

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, (1989) or Current Protocols in Molecular Biology Volumes 1-3 (Ausubel, et al., eds. 1994-1998).

The isolation of nucleic acids corresponding to target genes may be accomplished by a number of techniques. For instance, oligonucleotide probes based on known sequences can be used to identify the desired gene in a CDNA or genomic DNA library. To construct genomic libraries, large segments of genomic DNA are generated by random fragmentation, e.g. using restriction endonucleases, and are ligated with vector DNA to form concatamers that can be packaged into the appropriate vector. To prepare a cDNA library, mRNA is isolated from the desired organ, such as flowers, and a cDNA library which contains the target gene transcript is prepared from the mRNA. Alternatively, cDNA may be prepared from mRNA extracted from other tissues in which target genes or homologs are expressed.

The cDNA or genomic library can then be screened using a probe based upon the sequence of a cloned target gene. Probes may be used to hybridize with genomic DNA or cDNA sequences to isolate homologous genes in the same or different plant species. Alternatively, antibodies raised against an target polypeptide can be used to screen an mRNA expression library.

Alternatively, the nucleic acids of interest can be amplified from nucleic acid samples using amplification techniques. For instance, polymerase chain reaction (PCR) technology can be used to amplify the sequences of the target genes directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired MRNA in samples, for nucleic acid sequencing, or for other purposes. For a general overview of PCR see PCR Protocols: A Guide to Methods and Applications. (Innis et al., eds. 1990).

Polynucleotides may also be synthesized by well-known techniques as described in the technical literature (see, e.g., Carruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

Promoters and Expression Vectors

As described below, an improved gene silencing expression vector can be introduced into a plant by any suitable method. For example, the construct can be introduced into a plant via stable transformation with Agrobacterium, particle bombardment, electroporation, or transduction with a viral particle. A suitable expression vector is therefore selected according to the desired method of plant transformation.

In one embodiment, the construct is expressed via a DNA expression vector. Such expression vectors comprise DNA dependent RNA polymerase promoters that are active in plant cells, e.g., constitutive plant promoters such as those described herein and above (e.g., the nopaline synthase promoter, Sanders et al., Nuc. Acids Res. 15:1543-1558 (1987); or the CaMV 35S promoter, Urwin et al., Mol. Plant Microbe Interact. 10:394-400 (1997)) or tissue specific plant promoters such as those described herein and above.

In another embodiment, the construct is expressed via an RNA expression vector. The RNA expression vector encodes an RNA dependent RNA polymerase active in plant cells, and the construct is transcribed via an RNA dependent RNA polymerase promoter active in plant cells. Suitable RNA dependent RNA polymerases and their corresponding promoters and expression vectors are derived, e.g., from potato virus X (Chapman et al., Plant J. 2:549-557 (1992), tobacco mosaic virus (see, e.g., Dawson et al., Virology 172:285-292 (1989)), tobacco etch virus (see, e.g., Dolja et al., Proc. Nat'l Acad. Sci. USA 89:10208-10212 (1992)), tobacco rattle virus (see, e.g., Ziegler-Graff et al., Virology 182:145-155 (1991)), tomato bushy stunt virus (see, e.g., Scholthof et al., Mol. Plant Microbe Interact. 6:309-322 (1993)), brome mosaic virus (see, e.g., Mori et al., J Gen. Virol. 74:1255-1260 (1993)),. Such expression vectors are prepared using techniques known to those of skill in the art, e.g., by using bacterial RNA polymerases such as SP6 and T7 followed by manual inoculation, or by introduction of the vectors into plants by Agrobacterium-mediated transformation (Angell & Baulcombe, EMBO J. 16: 3675-3684 (1997)).

In another embodiment, optionally, a DNA expression vector also comprises a gene encoding an RNA dependent RNA polymerase active in plant cells. The RNA dependent RNA polymerase is then used to amplify the construct (either the positive and/or the negative strand).

In another embodiment, the construct is expressed via a DNA expression vector derived from a plant DNA virus, e.g., cauliflower mosaic virus (see, e.g., Futterer & Hohn, EMBO J. 10:3887-3896 (1991), African cassava mosaic virus (see, e.g., Ward et al., EMBO J. 7:1583-1587 (1988)) and the tomato golden mosaic virus.

In the present invention, a plant promoter may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill. Such genes include for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (GenBank No. X74782, Solocombe et al. Plant Physiol. 104:1167-1176 (1994)), GPc1 from maize (GenBank No. X15596, Martinez et al. J. Mol. Biol. 208:551-565 (1989)), and Gpc2 from maize (GenBank No. U45855, Manjunath et al., Plant Mol. Biol. 33:97-112 (1997)).

Alternatively, the plant promoter may direct expression of the transcript in a specific tissue, organ or cell type (i.e. tissue-specific promoters) or may be otherwise under more precise environmental or developmental control (i.e. inducible promoters). Examples of environmental conditions that may effect transcription by inducible promoters include pathogen challenge, anaerobic conditions, elevated temperature, the presence of light, or spraying with chemicals/hormones. One of skill will recognize that a tissue-specific promoter may drive expression of operably linked sequences in tissues other than the target tissue. Thus, as used herein a tissue-specific promoter is one that drives expression preferentially in the target tissue or cell type, but may also lead to some expression in other tissues as well.

A number of tissue-specific promoters can also be used in the invention. For instance, promoters that direct expression of nucleic acids in roots and feeding cells can be used. In particular, such promoters are useful for using the methods of the invention to inhibit nematode endoparasites that live in roots. The root-specific ANR1 promoter is suitable for use in the present invention (Zhang & Forde, Science 279:407 (1998)). The wound specific promoter wun-1 from potato can be used, as it respond to intracellular root migration by Globodera sp. (see, e.g., Hansen et al., Physiol. Mol. Plant Pathol. 48:161-170 (1996)). Other genes that demonstrate parasitic nematode feeding-cell specific expression have been reported, and their promoters are suitable for use in the present invention (see, e.g., Bird et al., Mol. Plant Microbe Interact. 7:419-424 (1994); Gurr et al., Mol. Gen. Genet. 226:361-366 (1991)); Lambert et al., Nucl. Acids. Res. 21:775-776 (1993); Opperman et al., Science 263:221-223 (1994); Van der Eycken et al., Plant J. 9:45-54 (1996); and Wilson et al., Phytopathology 84:299-303 (1992)). Phloem specific promoters, which can be used to transcribe the gene silencing construct of the invention for uptake by sap-sucking insects, include those referenced in Shi et al., J. Exp. Bot. 45:623-631 (1994).

The vector comprising the sequences of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Plant Transformation

Expression vectors of the invention may be introduced into the genome of the desired plant host by a variety of conventional techniques. For example, the expression vector may be introduced directly into the genomic DNA of the plant cell using techniques such as electroporation and microinjection of plant cell protoplasts, or the expression vectors can be introduced directly to plant tissue using ballistic methods, such as particle bombardment. In addition, the constructs of the invention may be introduced in plant cells as DNA or RNA expression vectors or viral particles that co-express an RNA dependent RNA polymerase.

Microinjection techniques are known in the art and well described in the scientific and patent literature. The introduction of expression vectors using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Alternatively, the expression vectors may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature (see, e.g., Horsch et al., Science 233:496-498 (1984); Fraley et al. Proc. Natl. Acad. Sci. USA 80:4803 (1983) and Gene Transfer to Plants (Potrykus, ed. 1995)).

Transformed plant cells which are derived by any of the above transformation techniques can be cultured to regenerate a whole plant which possesses the transformed genotype and thus the desired phenotype such as enhanced resistance to pathogens. Such regeneration techniques rely on manipulation of certain phytohornones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176 (1983); and Binding, Regeneration of Plants, Plant Protoplasts, pp. 21-73 (1985). Regeneration can also be obtained from plant callus, explants, organs, or parts thereof. Such regeneration techniques are described generally in Klee et al., Ann. Rev. of Plant Phys. 38:467-486 (1987).

The nucleic acids of the invention can be used to confer desired traits on essentially any plant. Thus, the invention has use over a broad range of plants, including wheat, corn, rice, sorghum, pepper, tomato, squash, banana, strawberry, carrot, bean, cabbage, beet, cotton, grape, pea, pineapple, potato, soybean, yam, and alfalfa, as well as other species described herein.

One of skill will recognize that after the expression cassette is stably incorporated in transgenic plants, if such a technique is used, and confirmed to be operable, it can be introduced into other plants by sexual crossing. Any of a number of standard breeding techniques can be used, depending upon the species to be crossed.

Using known procedures one of skill can screen for plants of the invention by detecting the effect of the construct of the invention in the target organism, either using in vitro assays such as plant culture, or in vivo assays such as transgenic plants. Means for directly and indirectly detecting and quantitating protein and RNA expression in vitro and in cells are well known in the art.

All publications, patents, and patent applications cited in this specification are herein incorporated by reference as if each individual publication, patent or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

In the example described below, a construct containing a poly-dT sequence is described, which allows insertion of any targeting sequence of interest 5′ to the poly-dT sequence. The poly-dT sequence is positioned 5′ to the transgene polyadenylation site in the orientation that specifies the incorporation of poly-U in the RNA transcript. The poly-A tail of the mRNA transcript can form a double-stranded structure with the poly-U region and target the transcript for degradation. Gene silencing is thus accomplished by interaction of the poly-U region, specified by the construct, and the poly-A tail, which is added as part of the normal process of mRNA production in eukaryotic cells.

Unless otherwise indicated, all procedures and methodologies described herein are described in the molecular biology methods handbook of Sambrook et al., Molecular Cloning (1990), and enzymes and chemicals used in DNA manipulation were purchased from suppliers including New England Bioblabs, Stratagene, Promega, Life Technologies, Clontech, Invitrogen, Qiagen, and Sigma.

The first step of the poly dT silencing vector development involves cloning a poly[dT].poly[dA] homopolymeric double stranded sequence into a vector with suitable restriction sites flanking the poly[dT].poly[dA] region. Plasmid pSport1 (Life Technologies) is digested to completion with SpeI and XbaI, dephosphorylated with Calf Intestinal Alkaline Phosphatase and the ends filled in with Klenow. Approximately 5 micrograms of double stranded homopolymeric polydeoxyadenylic acid-polythymidylic acid is run on an agarose gel alongside DNA molecular weight markers in an adjacent lane. DNA is visualized by ethidium bromide fluorescence using a UV light of approximately 312 nm and the poly[dT].poly[dA] material migrating in the 100 bp-1000 bp range is excised and extracted from the gel using the Qiagen gel extraction kit. The ends of this material are made flush by treatment with T4 DNA polymerase in the presence of 100 micromolar dATP and dTTP. The filled in, size selected homopolymeric poly[dT].poly[dA] population is ligated with the SpeI+XbaI digested, filled in pSport1.

The reaction is used to transform E. coli DH5α to ampicillin resistance on LB+amp plates containing IPTG and X-Gal as an indicator of beta-galactosidase activity. White colonies are picked and plasmid DNA prepared by the boiling method. To ascertain whether an insert has been cloned, plasmids are digested with XbaI and SpeI and the products resolved on an agarose gel. Plasmids containing inserts in the 50-1100 bp range are further characterized by sequencing the inserts. Plasmids containing from about 50 to about 1100 dT residues inserted between the XbaI and SpeI sites and in the desired orientation are used for further constructs.

The poly[dT] sequence is removed from several of these plasmids by digestion with XbaI and SalI, resolving the products on an agarose gel and isolating the poly dT fragment using the Qiagen gel extraction kit. The poly dT fragment is ligated to the 4389 bp gel purified fragment derived from XbaI+SalI digestion of pPO7212 and the reaction is used transform E. coli DH5α to ampicillin resistance. Colonies are picked, plasmid DNA prepared by the boiling method, digested with XbaI+SalI and resolved on an agarose gel to verify that the poly [dT] fragment is present. A particular poly dT silencing plasmid constructed in this manner (see corresponding schematic, FIG. 1), contains the enhanced 35S promoter of the CaMV followed by the petunia Cab22 5′ leader followed by unique NcoI and SpeI sites followed by the homopolymeric poly[dT] sequence followed by the 3′ untranslated region of the Agrobacterium octopine synthase gene in the standard plasmid vector pUC19. The length of the poly dT sequence will vary from one plasmid to another depending on the size initially cloned however this length will be specific for a given plasmid. Upon insertion of any desired sequence in the Nco and SpeI sites, the transgene can be moved to an appropriate plasmid by digestion with BamHI and BglII and introduced into plants by standard methods.

To assess the ability of targeting sequences in such poly dT silencing plasmids to suppress the expression of endogenous genes and transgenes two types of constructs are made. To assess the suppression of an endogenous gene, a portion of the Arabidopsis CONSTANS (CO) gene (GenBank Accession X94937) is isolated from Arabidopsis plants and cloned into the NcoI and SpeI sites of several poly dT silencing plasmids that differ in the lengths of the poly dT sequence. RNA is isolated from developing inflorescence tissue using the RNeasy kit from Qiagen according to the manufacturers protocol. First strand cDNA is prepared using the SuperScript™ Preamplification System from Life Technologies. A 714 bp PCR product corresponding to nucleotides 198 to 912 of the Arabidopsis CO transcript (see FIG. 2 of Putterill et al., Cell 80:847-857 (1995)) is generated using primers NC626 (5′ CGCTCATGAGCGTGCTCCGGCTGCTTT 3′) and NC627 (5′ CGCACTAGTCATTGGACTGAGTTGTGTTACTGTT 3′) in an RT-PCR using standard reaction conditions and a thermocycling regime composed of 35 cycles of 30 sec @ 94° C., 30 sec @ 60° C. and 60 sec @ 72° C. The product is digested with BspHI and SpeI, then ligated individually with several poly dT silencing plasmids previously digested with NcoI and SpeI.

The reactions are used to transform E. coli DH5α to ampicillin resistance. Colonies are picked, plasmid DNA prepared by the boiling method, digested with NcoI+SpeI and the products resolved on an agarose gel to verify that the Arabidopsis CONSTANS fragment is cloned. Plasmids are digested with BglII+BamHI, the fragments containing the CaMV35S/Cab22/CO/poly dT/OCS transgene are gel purified and ligated with binary plasmid pBIN19 (Bevan, Nucleic Acids Res. 12:8711 (1984)) previously digested with BamHI. Plasmid pBin19 is suitable for replication in E. coli as well as Agrobacterium hosts; it confers kanamycin resistance to both of these bacterial hosts and contains a T-DNA region harboring a selectable marker gene that confers to plant cells resistance to kanamycin. The ligation reactions are used to transform E. coli DH5α to kanamycin resistance. Colonies are picked, and plasmid DNA is prepared by the boiling method and digested with a series of restriction enzymes to verify the integrity of the binary plasmid.

To assess the suppression of a transgene gene, the GUS reporter gene (E. coli uidA or GUS gene, Genbank Accession S69414) is substituted for the Arabidopsis CO gene fragment in the preceding poly dT silencing plasmids. Primers 626 (5′ CCATGGGTCAGTCCCTTATGTTACG 3′and 627 (5′ GACTCTAGATCATTGTTGCCTCCCTGCTGCGG 3′are used to amplify the coding region of the GUS gene in a PCR using standard conditions and a thermocycling regime composed of 35 cycles of 95° C. for 30 sec, 55° C. for 30 sec and 72° C. for 60 sec. Any source of the GUS gene (e.g., plasmid pRAJ220, Jefferson et al., Proc. Nat'l Acad. Sci. USA 83:8447-8451 (1986)) can be used as the template in this reaction.

The PCR product is digested with NcoI+XbaI and ligated with poly dT silencing plasmids previously digested with NcoI and SpeI and the reactions used to transform E. coli DH5α to ampicillin resistance. Colonies are picked; plasmid DNA is prepared by the boiling method and digested with NcoI+XbaI; and the products resolved on an agarose gel to verify that the GUS gene fragment is cloned. Plasmids are digested with BglII+BamHI, the fragments containing the CaMV35S/Cab22/GUS/poly dT/OCS transgene are gel purified and ligated with binary plasmid pBin19 previously digested with BamHI. Plasmid pBin19 is suitable for replication in E. coli as well as Agrobacterium hosts; it confers kanamycin resistance to both of these bacterial hosts and contains a T-DNA region harboring a selectable marker gene that confers to plant cells resistance to kanamycin. The ligation reactions are used to transform E.coli DH5α to kanamycin resistance. Colonies are picked, and plasmid DNA is prepared by the boiling method and digested with a series of restriction enzymes to verify the integrity of the binary plasmid.

Arabidopsis plants are transformed with GUS/poly-dT and CO/poly-dT silencing constructs, using the floral dip method described by Clough & Bent (Plant J. 16(6):735-743 (1998)). Binary plasmids containing the poly dT silencing transgenes described above are transformed into an Agrobacterium strain suitable for transformation of Arabidopsis thaliana (e.g., LBA4404) and cultures used to transform suitable Arabidopsis plants as described (Clough & Bent (1998), supra). For suppression of Arabidopsis CO, wildtype plants are used. For the suppression of the GUS reporter gene, plants harboring a constitutively transcribed GUS transgene (such as a Cauliflower Mosaic Virus 35S promoter fused to the GUS coding region and a nopaline synthase 3′ untranslated region) are used. Suppression is judged to occur in plants harboring the poly dT silencing transgene if the activity of the targeted gene is measured to be less than in plants without the poly dT silencing transgene.

Plants transformed with the CO/poly dT silencing transgene that exhibit reduced activity may be identified based on their abnormal time of flowering compared to wildtype plants. Plants with reduced CO activity will require longer time to flower when grown under long day lengths compared to wildtype plants. Plants harboring a constitutively transcribed GUS transgene which have been transformed with the GUS/poly dT silencing transgene and exhibit reduced activity of the GUS protein are identified as having lowered activity in a standard GUS enzyme assay when compared with plants harboring the constitutively transcribed GUS transgene but not the GUS/poly dT silencing transgene. Alternatively, plants that exhibit silencing acan be identified based on lower levels of mRNA of the targeted gene using northern blots, primer extension, RT-PCR, or any other suitable mRNA quantification method.

What is claimed is:

1. A method of reducing expression of a target gene in a eukaryotic cell, the method comprising the step of expressing in the cell an expression cassette comprising a promoter operably linked to a sense or antisense targeting sequence having substantial identity to at least a subsequence of the target gene, a poly-dT sequence, and a polyadenylation signal, wherein the poly-dT sequence is positioned 5′ to the polyadenylation signal, thereby reducing expression of the target gene.

2. The method of claim 1, wherein the poly-dT sequence is positioned 3′ to the targeting sequence.

3. The method of claim 1, wherein the poly-dT sequence is positioned 5′ to the targeting sequence.

4. The method of claim 1, wherein a linker sequence is present between the poly-dT sequence and the polyadenylation signal.

5. The method of claim 1, wherein the poly-dT sequence is from about 50 to about 1000 nucleotides in length.

6. The method of claim 1, wherein the targeting sequence is a sense sequence.

7. The method of claim 1, wherein the targeting sequence is an antisense sequence.

8. The method of claim 1, wherein the targeting sequence has substantial identity to a plant pathogen target gene.

9. The method of claim 8, wherein the targeting sequence is a viral sequence, a bacterial sequence, an insect sequence, a fungal sequence, a parasitic sequence, or a nematode sequence.

10. The method of claim 1, wherein the targeting sequence has substantial identity to a plant target gene.

11. The method of claim 1, wherein the targeting sequence is from about 100 to about 1000 nucleotides in length.

12. The method of claim 1, wherein the targeting sequence is from a coding region of the target gene.

13. The method of claim 1, wherein the targeting sequence is from a 5′ untranslated region of the target gene.

14. The method of claim 1, wherein the targeting sequence is from a 3′ untranslated region of the target gene.

15. The method of claim 1, wherein the target gene is CONSTANS.

16. The method of claim 1, wherein the promoter is a tissue specific promoter.

17. The method of claim 1, wherein the promoter is a plant promoter.

18. The method of claim 17, wherein the promoter is a cauliflower mosaic virus 35S promoter or a figwort mosaic virus 34S promoter.

19. The method of claim 1, wherein the cell is a plant cell.

20. The method of claim 19, wherein the plant is selected from the group consisting of wheat, corn, rice, sorghum, pepper, tomato, squash, banana, strawberry, carrot, bean, cabbage, beet, cotton, grape, pea, pineapple, potato, soybean, yam, and alfalfa.

21. The method of claim 1, wherein the targeting sequence comprises a premature stop codon that inhibits translation of the targeting sequence.

22. An expression cassette comprising a promoter operably linked to a sense or antisense targeting sequence having substantial identity to at least a subsequence of the target gene, a poly-dT sequence, and a polyadenylation signal, wherein the poly-dT sequence is positioned 5′ to the polyadenylation signal.

23. The expression cassette of claim 22, wherein the poly-dT sequence is positioned 3′ to the targeting sequence.

24. The expression cassette of claim 22, wherein the poly-dT sequence is positioned 5′ to the targeting sequence.

25. The expression cassette of claim 22, wherein a linker sequence is present between the poly-dT sequence and the polyadenylation signal.

26. The expression cassette of claim 22, wherein the poly-dT sequence is from about 50 to about 1000 nucleotides in length.

27. The expression cassette of claim 22, wherein the targeting sequence is a sense sequence.

28. The expression cassette of claim 22, wherein the targeting sequence is an antisense sequence.

29. The expression cassette of claim 22, wherein the targeting sequence has substantial identity to a plant pathogen target gene.

30. The expression cassette of claim 29, wherein the targeting sequence is a viral sequence, a bacterial sequence, an insect sequence, a fungal sequence, a parasitic sequence, or a nematode sequence.

31. The expression cassette of claim 22, wherein the targeting sequence has substantial identity to a plant target gene.

32. The expression cassette of claim 22, wherein the targeting sequence is from about 100 to about 1000 nucleotides in length.

33. The expression cassette of claim 22, wherein the targeting sequence is from a coding region of the target gene.

34. The expression cassette of claim 22, wherein the targeting sequence is from a 5′ untranslated region of the target gene.

35. The expression cassette of claim 22, wherein the targeting sequence is from a 3′ untranslated region of the target gene.

36. The expression cassette of claim 22, wherein the target gene is CONSTANS.

37. The expression cassette of claim 22, wherein the promoter is a tissue specific promoter.

38. The expression cassette of claim 22, wherein the promoter is a plant promoter.

39. The expression cassette of claim 38, wherein the promoter is a cauliflower mosaic virus 35S promoter or a figwort mosaic virus 34S promoter.

40. The expression cassette of claim 22, wherein the cell is a plant cell.

41. The expression cassette of claim 40, wherein the plant is selected from the group consisting of wheat, corn, rice, sorghum, pepper, tomato, squash, banana, strawberry, carrot, bean, cabbage, beet, cotton, grape, pea, pineapple, potato, soybean, yam, and alfalfa.

42. The expression cassette of claim 22, wherein the targeting sequence comprises a premature stop codon that inhibits translation of the targeting sequence.

43. A transgenic plant comprising the expression cassette of claim 22.
